(19) United States
(12) Patent Application Publication
(10) Pub. No.: US 2008/0045572 A1
(43) Pub. Date: Feb. 21, 2008

(54) Pharmaceutical Composition Comprising Roflumilast or the N-Oxide of Roflumilast

(75) Inventor: Rudolf Linder, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 11/662,888

(22) PCT Filed: Sep. 21, 2005
(86) PCT No.: PCT/EP05/54724

§ 371 Date: Mar. 15, 2007

(30) Foreign Application Priority Data
Sep. 22, 2004 (DE) 102004046236.4

(51) Int. Cl. A61K 31/44; A61K 31/164; A61P 43/00

(52) U.S. Cl. 514/352; 514/617

(57) ABSTRACT

An aqueous pharmaceutical preparation for administration of a slightly soluble PDE4 inhibitor is described.

### TECHNICAL FIELD

The present invention relates to the field of pharmaceutical technology and describes an aqueous pharmaceutical preparation comprising a slightly soluble PDE 4 inhibitor as active ingredient. The invention further relates also to processes for producing the pharmaceutical preparation and to the use of the pharmaceutical preparation for the treatment of disorders.

### PRIOR ART

Cyclic nucleotide phosphodiesterase (PDE) inhibitors (in particular of type 4) are currently of particular interest as a new generation of active ingredients for the treatment of inflammatory disorders, especially disorders of the respiratory tract such as asthma or airway obstructions (such as, for example, COPD=chronic obstructive pulmonary disease). A number of PDE 4 inhibitors is currently undergoing advanced clinical trials, including a dosage form for oral administration comprising the active ingredient N-(3,5-dichloropyrid-4-yl)-3-cyclopropylmethoxy-4-difluoromethoxybenzamide (INN: roflumilast). This and other compounds having a benzamide structure and their use as cyclic nucleotide phosphodiesterase (PDE) inhibitors are described in WO95/01338. These active ingredients are proposed in WO95/01338 also for the treatment of certain disorders of the skin (such as, for example, dermatoses). WO00/53182 proposes the use of roflumilast or its N-oxide for the treatment of multiple sclerosis. WO03/099334 describes pharmaceutical preparations for slightly soluble PDE 4 inhibitors such as, for example, ointments and aqueous or oily suspensions and emulsions.

Besides oral dosage forms, it may also be necessary and advantageous to provide an active ingredient as parenteral form (preparation intended for injection), especially for groups of patients who have problems with taking oral dosage forms.

Parenteral preparations must be produced with particular care in order to guarantee freedom from irritation and to avoid microbial and particulate contaminants. The most important solvent or dispersant is water. According to the pharmacopeoia, water for injections must always be used in these cases. Active ingredients to be administered intravenously must be completely dissolved. It must furthermore be ensured during development of the preparation that no precipitation takes place during the injection. Further requirements to be mentioned for parenteral preparations are in particular good tolerability for the patient. This may depend on the aqueous preparation being rendered isotonic or approximately isotonic and having an approximately physiological pH, and on the absence of particulate contaminants.

The production of parenteral preparations or of general solutions of active ingredients which are slightly soluble in water therefore involves particular problems. For example, for the PDE 4 inhibitor N-(3,5-dichloropyrid-4-yl)-3-cyclopropylmethoxy-4-difluoromethoxybenzamide (INN: roflumilast) described in WO95/01338 the solubility in water is observed to be only 0.53 mg/l at 21° C. Replacement of water by other solvents or the use of solubilizers [e.g. solubilizers such as lecithin and poloxamer 188 (Pluronic F68®)] is, however, possible to only a limited extent for producing parenteral preparations or solutions for other purposes for the reasons indicated above. An attempt to produce an aqueous solution of roflumilast, e.g. for injection, does not, despite addition of the solubilizer poloxomer 188 which is normally employed in connection with parenteral preparations, result in a solution having a roflumilast concentration which is acceptable for injections.

### DESCRIPTION OF THE INVENTION

It has now been found, surprisingly, that roflumilast can be dissolved in water in an amount which is sufficient for injections when the solution has an alkaline pH. It is possible on this basis to obtain clear solutions having the properties necessary for parenteral preparations (in particular good tolerability for the patient, no particulate contaminants). In particular, the solution is also stable during storage and no precipitates of the active ingredient are observed. Furthermore, compatibility with the container material is good.

The invention therefore relates to an aqueous pharmaceutical preparation comprising an active ingredient in a therapeutically effective and pharmacologically acceptable amount, where the active ingredient is selected from the group consisting of roflumilast, salts of roflumilast, the N-oxide of the pyridine residue of roflumilast or salts thereof and where the solution has an alkaline pH. The aqueous preparation is in particular a solution in which the active ingredient is completely dissolved.

Roflumilast is the INN for a compound of the formula I 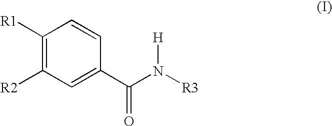
in which
R1 is difluoromethoxy,
R2 is cyclopropylmethoxy and
R3 is 3,5-dichloropyrid-4-yl.

This compound has the chemical name N-(3,5-dichloropyrid-4-yl)-3-cyclopropylmethoxy-4-difluoromethoxybenzamide (INN: roflumilast). The N-oxide of roflumilast has the chemical name 3-cyclopropylmethoxy-4-difluoromethoxy-N-(3,5-dichloropyrid-4-yl 1-oxide)benzamide.

This compound of the formula I, its salts, the N-oxide, its salts and the use of these compounds as phosphodiesterase (PDE) 4 inhibitors are described in the International Patent Application WO95/01338.

Salts suitable for compounds of the formula I—depending on the substitution—are all acid addition salts but, in particular, all salts with bases. Particular mention may be made of the pharmacologically acceptable salts of the inorganic and organic acids and bases normally used in pharmaceutical technology. Pharmacologically unacceptable salts which, for example, may be the initial products of the process for preparing the compounds of the invention on the industrial scale are converted into pharmacologically acceptable salts by processes known to the skilled worker. Those suitable on the one hand are water-soluble and water-insoluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid, or 3-hydroxy-2-naphthoic acid, the acids being employed to prepare the salts in the equimolar ratio of amounts, or one differing therefrom—depending on whether the acid is monobasic or polybasic and depending on which salt is desired.

On the other hand, salts with bases are also particularly suitable. Examples of basic salts which may be mentioned are lithium, sodium, potassium, calcium, magnesium, ammonium, meglumine or guanidinium salts, once again the bases being employed to prepare the salts in the equimolar ratio of amounts or one differing therefrom.

An alkaline pH of the solution means according to the invention a pH of greater than 7, in particular greater than or equal to 8. The solution according to the invention preferably has an alkaline pH in the range from pH 10 to pH 13, preferably pH 10.5 to pH 12.5 and very particularly preferably pH 11 to pH 12.3. An alkaline pH is adjusted for the solution according to the invention preferably by adding a suitable compound with an alkaline reaction (base). Particularly suitable compounds with an alkaline reaction are according to the invention sodium hydroxide and/or sodium carbonate. The compound with an alkaline reaction is in this connection added to the solution according to the invention in an amount such that the desired pH is adjusted.

The preparation according to the invention is preferably a preparation based on water, especially water for injection.

The preparation according to the invention may if desired comprise further pharmaceutical excipients suitable for parenteral preparations. Examples which should be mentioned here are substances to improve the solubility (e.g. cosolvents and solubilizers), substances for rendering isotonic, buffers, antioxidants, chelating agents, preservatives, emulsifiers, organic acids, salts of organic acids or excipients to prolong the effect.

Suitable cosolvents are in particular ethanol, glycerol, propylene glycol and polyethylene glycol or 1,3-butanediol. Preference is given in this connection to propylene glycol and polyethylene glycol (especially macrogol 300/400). Examples of solubilizers which should be mentioned are lecithin and poloxamer 188 (Pluronic F68®), with preference for poloxamer 188.

Substances which are used for rendering isotonic and should be mentioned in particular are sodium chloride, glucose, mannitol, glycerol, propylene glycol and polyethylene glycol.

Organic acids and/or salts thereof which may be mentioned in particular are glycine, lysine, acetic acid, sodium acetate or citric acid. The organic acid and/or salt thereof is preferably employed in an amount such that the molar ratio of roflumilast to organic acid and/or the salts is in the range from 1:1 to 1:2.

A solubilizer which may be mentioned in particular is polyvinylpyrrolidone (e.g. Kollidon 12 or 17® from BASF).

100 ml of solution comprise from 0.1 mg to 0.9 g of active ingredient, preferably 0.5 mg to 0.85 g, very particularly preferably 1 mg to 0.7 g. Poloxamer 188 (Pluronic F 68®) can be employed in amounts of from 0.1 g to 10 g.

The pharmaceutical preparation according to the invention can be produced by processes familiar to the skilled person. The active ingredient is preferably suspended in water, it being possible to add if desired also a solubilizer such as poloxamer 188 and/or an organic acid and/or salt thereof. It is additionally possible also to add further excipients suitable for parenteral preparations, such as agents for rendering isotonic or cosolvents. The preparation is then adjusted to the desired pH by adding the compound with an alkaline reaction, if desired with heating. A clear solution is obtained. The solution obtained in this way can then be sterilized by filtration and subsequently dispensed into suitable containers such as vials or ampoules. The solution can alternatively first be introduced into suitable containers, and this solution can then be subjected in the final container to a sterilization, for example by autoclaving.

The following examples serve to illustrate the invention without restricting it.

Production of the Dosage Forms According to the Invention

### EXAMPLE 1

| Roflumilast | 0.08 | g |
|---|---|---|
| Glycine | 0.04 | g |
| Poloxamer 188 | 1.00 | g |
| Sodium hydroxide | q.s. to pH 11-12 | |
| Glycerol | 2.50 | g |

| | |
|---|---|
| Water for injections | to 100 ml |

A clear solution of the poloxamer 188, glycerol and the glycine in water is prepared. The active ingredient is added, and the mixture is heated to about 80° C. Dilute sodium hydroxide solution is added until a pH of 11-12 is reached and the active ingredient has formed a clear solution. The solution is sterilized by filtration, dispensed into ampoules and sterilized.

### EXAMPLE 2

| | | |
|---|---|---|
| Roflumilast | 0.01 | g |
| Polyethylene glycol 400 | 10.00 | g |
| Poloxamer 188 | 0.50 | g |

| | |
|---|---|
| Sodium hydroxide | q.s. to pH 11-12 |
| Water for injections | to 100 ml |

A clear solution of the poloxamer 188 and the polyethylene glycol 400 in water is prepared. The active ingredient is dispersed therein, and the preparation is heated to about 80° C. The active ingredient is dissolved and the pH is adjusted to 11-12 by adding sodium hydroxide solution. The solution is sterilized by filtration, dispensed into ampoules and sterilized.

### EXAMPLE 3

| | | |
|---|---|---|
| Roflumilast | 0.05 | g |
| Sodium acetate | 0.02 | g |
| Poloxamer 188 | 2.00 | g |
| Propylene glycol | 5.00 | g |

| | |
|---|---|
| Sodium hydroxide | q.s. to pH 11-13 |
| Water for injections | to 100 ml |

A clear solution of the poloxamer 188, sodium acetate and propylene glycol in water is prepared. The solution is heated to about 80° C. and the active ingredient is dispersed. The active ingredient is dissolved and the pH is adjusted to 11-13 with sodium hydroxide solution. The solution is sterilized by filtration and dispensed under aseptic conditions into ampoules.

### EXAMPLE 4

| | | |
|---|---|---|
| Roflumilast | 0.01 | g |
| Polyethylene glycol 400 | 10.00 | g |

| | |
|---|---|
| Sodium hydroxide | q.s. to pH 11-12 |
| Water for injections | to 100 ml |

The roflumilast is stirred into 10 grams of polyethylene glycol 400. 70 ml of water for injections are added, and a clear solution of the active ingredient is prepared with dilute sodium hydroxide solution. The pH is then about 11-12. The clear solution is made up to 100 ml, sterilized by filtration, dispensed into ampoules and sterilized.

### EXAMPLE 5

| | | |
|---|---|---|
| Roflumilast | 0.08 | g |
| Polyethylene glycol 400 | 10.00 | g |
| Glycine | 0.04 | g |

| | |
|---|---|
| Sodium hydroxide | q.s. to pH 11-12 |
| Water for injections | to 100 ml |

A clear solution of the glycine and the polyethylene glycol 400 in water is prepared. The active ingredient is added and the solution is heated to about 80° C. Dilute sodium hydroxide solution is added until a pH of 11-12 is reached and a clear solution of the active ingredient has formed. The solution is made up to 100 ml, sterilized by filtration, dispensed into ampoules and sterilized.

### INDUSTRIAL APPLICABILITY

The preparations of the invention can be employed for the treatment and prevention of all diseases regarded as treatable or preventable through the use of PDE 4 inhibitors. Selective cyclic nucleotide phosphodiesterase (PDE) inhibitors (specifically of type 4) are suitable on the one hand as bronchial therapeutic agents (for the treatment of airway obstructions owing to their dilating effect but also owing to their effect increasing the respiratory rate and respiratory drive) and for eliminating erectile dysfunction owing to the vasodilating effect, but on the other hand especially for the treatment of disorders, especially of an inflammatory nature, e.g. of the airways (asthma prophylaxis), of the skin, of the central nervous system, of the intestine, of the eyes and of the joints, which are promoted by mediators such as histamine, PAF (platelet-activating factor), arachidonic acid derivatives such as leukotrienes and prostaglandins, cytokines, interleukins, chemokines, alpha-, beta- and gamma-interferon, tumour necrosis factor (TNF) or oxygen free radicals and proteases. The pharmaceutical preparations of the invention can therefore be used in human and veterinary medicine for example for the treatment and prophylaxis of the following diseases: acute and chronic (especially inflammatory and allergen-induced) airway disorders of various aetiologies (bronchitis, allergic bronchitis, bronchial asthma, COPD); dermatoses (especially of a proliferative, inflammatory and allergic nature) such as, for example, psoriasis (vulgaris), toxic and allergic contact eczema, atopic eczema, seborrhoeic eczema, lichen simplex, sunburn, pruritus in the genitoanal region, alopecia greata, hypertrophic scars, discoid lupus erythematosus, follicular and extensive pyodermas, endogenous and exogenous acne, acne rosacea and other proliferative, inflammatory and allergic skin disorders; disorders based on excessive release of TNF and leukotrienes, e.g. disorders of the arthritic type (rheumatoid arthritis, rheumatoid spondylitis, osteoarthritis and other arthritic states), disorders of the immune system (AIDS, multiple sclerosis), types of shock [septic shock, endotoxin shock, Gram-negative sepsis, toxic shock syndrome and ARDS (adult respiratory distress syndrome)] and generalized inflammations in the gastrointestinal region (Crohn's disease and ulcerative colitis); disorders based on allergic and/or chronic abnormal immunological reactions in the region of the upper airways (pharyngeal space, nose) and adjacent regions (paranasal sinuses, eyes), such as, for example, allergic rhinitis/sinusitis, chronic rhinitis/sinusitis, allergic conjunctivitis, conjunctivitis caused by bacteria, viruses or fungi, inflammatory states after intraocular lens implantation, inflammation of the optic nerve (neuritis nervi optici), keratitis, dry eye syndrome (keratitis sicca), uveitis, glaucoma, retinal oedema, retinitis pigmentosa, diabetic retinopathy, and nasal polyps; but also cardiac disorders which can be treated by PDE inhibitors, such as, for example, heart failure, or disorders which can be treated owing to the tissue-relaxant effect of PDE inhibitors, such as, for example, erectile dysfunction or colic of the kidneys and ureters connected with kidney stones; or else disorders of the CNS such as, for example, depressions or arteriosclerotic dementia.

The pharmaceutical preparations of the invention are also suitable for the treatment of disorders of the skin such as dermatoses (especially of a proliferative, inflammatory and allergic nature) such as, for example, psoriasis (vulgaris), toxic and allergic contact eczema, atopic eczema, seborrhoeic eczema, lichen simplex, sunburn, pruritus in the genitoanal region, alopecia greata, hypertrophic scars, discoid lupus erythematosus, follicular and extensive pyodermas, endogenous and exogenous acne, acne rosacea and other proliferative, inflammatory and allergic skin disorders. The preparations according to the invention in the form of a solution can be administered parenterally (e.g. as injection or infusion) for the treatment of the abovementioned diseases.

The invention further relates to a method for the treatment of mammals, including humans, suffering from one of the abovementioned diseases. The method is characterized in that a therapeutically effective and pharmacologically suitable amount of an active pharmaceutical ingredient selected from the group of compounds roflumilast, salts of roflumilast, the N-oxide of roflumilast and salts thereof is administered to the mammal with the disease, with the active pharmaceutical ingredient being administered in a pharmaceutical preparation of the invention. The method is characterized in that the administration takes place by parenteral administration (injection or infusion).

The pharmaceutical preparations of the invention are moreover particularly suitable for administration to groups of patients who are suffering from the abovementioned diseases and have problems in taking pharmaceutical preparations to be administered orally, such as, for example, bedridden patients, patients in intensive medical care, patients with swallowing difficulties and children.

The invention further relates to a method for the treatment of mammals, including humans, suffering from one of the abovementioned diseases. The method is characterized in that a therapeutically effective and pharmacologically suitable amount of an active pharmaceutical ingredient selected from the group of compounds roflumilast, salts of roflumilast, the N-oxide of roflumilast and salts thereof is administered to the mammal with the disease, with the active pharmaceutical ingredient being administered in a topical pharmaceutical preparation of the invention.

The disease is preferably acute and chronic (especially inflammatory and allergen-induced) airway disorders of various aetiologies (bronchitis, allergic bronchitis, bronchial asthma, COPD), and disorders of the arthritic type (rheumatoid arthritis, rheumatoid spondylitis, osteoarthritis and other arthritic states).

The dosage forms of the invention comprise the active pharmaceutical ingredient in the dose customary for the treatment of the particular disease. The dosage of the active ingredient is of the order of magnitude customary for PDE inhibitors, it being possible to administer the daily dose in one or more dosage units. Customary dosages are disclosed for example in WO 95/01338. The normal dose on systemic therapy (oral) is between 0.001 and 3 mg per kilogram and day. Dosage forms preferred according to the invention for parenteral administration contain from 0.005 mg to 5 mg of roflumilast, preferably from 0.01 mg to 2.5 mg, particularly preferably 0.1 mg to 0.5 mg of roflumilast per dosage unit. Examples of pharmaceutical preparations of the invention contain 0.01 mg, 0.1 mg, 0.125 mg, 0.25 mg and 0.5 mg of roflumilast per dosage unit.

1. An aqueous pharmaceutical preparation comprising an active ingredient in a therapeutically effective and pharmacologically acceptable amount, where the active ingredient is selected from the group consisting of roflumilast, salts of roflumilast, the N-oxide of the pyridine residue of roflumilast and salts thereof and where the solution has an alkaline pH.

2. The aqueous pharmaceutical preparation according to claim 1, where roflumilast is a compound of the formula I 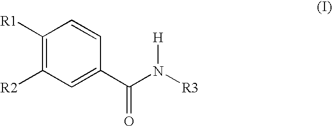
in which
R1 is difluoromethoxy,
R2 is cyclopropylmethoxy and
R3 is 3,5-dichloropyrid-4-yl.

3. The pharmaceutical preparation according to claim 1, which is a preparation for injection.

4. The pharmaceutical preparation according to claim 1, which is a clear solution.

5. The pharmaceutical preparation according to claim 1, which is an isotonic solution.

6. The pharmaceutical preparation according to claim 1, comprising a compound with an alkaline reaction.

7. The pharmaceutical preparation according to claim 1, where the solution has a pH in the range from pH 10 to pH 13.

8. The pharmaceutical preparation according to claim 1, where the solution has a pH in the range from pH 10.5 to pH 12.5.

9. The pharmaceutical preparation according to claim 1, wherein the solution has a pH in the range from pH 11 to pH 12.3.

10. The pharmaceutical preparation according to claim 1, comprising sodium hydroxide and/or sodium carbonate.

11. The pharmaceutical preparation according to claim 1, where the preparation comprises one or more further excipients selected from the group consisting of substances for improving the solubility of the preparation, substances for rendering the preparation isotonic, buffers, antioxidants, chelating agents, preservatives, emulsifiers, organic acids, salts of organic acids and excipients for prolonging the effect.

12. The pharmaceutical preparation according to claim 11, wherein one or more substances selected from the group consisting of poloxamer 188, polyethylene glycol and propylene glycol are present.

13. The pharmaceutical preparation according to claim 12, where poloxamer 188 is present.

14. The pharmaceutical preparation according to claim 12, where at least one substance selected from the group consisting of organic acid and salts of an organic acid is present.

15. The pharmaceutical preparation according to claim 14, where the organic acid is a substance selected from the group consisting of glycine, lysine, acetic acid, sodium acetate and citric acid.

16. The pharmaceutical preparation according to claim 14, where the molar ratio of roflumilast to the organic acid and/or salts thereof is in the range from 1:1 to 1:2.

17. The pharmaceutical preparation according to claim 1, comprising poloxamer 188 and sodium hydroxide, where the preparation has a pH in the range from 11 to 12.

18. The pharmaceutical preparation according to claim 17, where a substance for rendering the preparation isotonic is additionally present.

19. The pharmaceutical preparation according to claim 1, where from 0.1 mg to 0.9 g of roflumilast is present in 100 ml solution for injection.

20. A process for producing a pharmaceutical preparation according to claim 1, comprising the steps of (a) suspending the active ingredient in water and (b) adding a compound with an alkaline reaction.

21. The process according to claim 20, where one or more pharmaceutical excipients suitable for parenteral preparations are added in step (a).

22. The process according to claim 20, where step (b) is carried out with heating.

23. The process according to claim 20, where the solution obtained in step (b) is sterilized by filtration and dispensed into suitable containers.

24. The pharmaceutical preparation according to claim 1, where from 0.5 mg to 0.85 g of roflumilast is present in 100 ml solution for injection.

25. The pharmaceutical preparation according to claim 1, where from 1 mg to 0.7 g of roflumilast is present in 100 ml solution for injection.
